(19) United States
(12) Patent Application Publication
(10) Pub. No.: US 2026/0347293 A1
(43) Pub. Date: Nov. 5, 2026

(54) METHOD FOR THE CONTROL OF PRESSURE IN A LOOP FOR THE PREPARATION OF METHANOL

(71) Applicant: Topsoe A/S, Kgs. Lyngby (DK)

(72) Inventors: Troels Juel FRIIS-CHRISTENSEN, Smørum (DK); Martin MØLLERHØJ, Birkerød (DK)

(21) Appl. No.: 19/111,838

(22) PCT Filed: Oct. 24, 2023
(86) PCT No.: PCT/EP2023/079563

§ 371 Date: Mar. 14, 2025

(30) Foreign Application Priority Data
Oct. 25, 2022 (DK) PA202200964

(51) Int. Cl. C07C 29/152; C07C 29/151

(52) U.S. Cl. CPC C07C 29/152; C07C 29/1518

(57) ABSTRACT

A method for the control of pressure in a loop for the preparation of methanol using an anti-surge control valve of a compressor and/or a compressor flow regulation valve for the recirculation of loop recirculation gas at variating flow supply of fresh synthesis gas is described.

The present invention is directed to the method for the control of pressure in a loop for the preparation of methanol. More particular, the invention makes use of the anti-surge control valve of a compressor and/or a compressor flow regulation valve for the recirculation of a methanol loop recirculation gas at variating flow of fresh methanol synthesis gas.

As an example, synthesis gas is conventionally prepared by subjecting hydrocarbon feed of natural gas or higher hydrocarbons to endothermic steam reforming reactions in a fired tubular steam reformer by contact with a steam reforming catalyst. The primary reformed gas is then fed into a secondary adiabatic reformer, wherein part of hydrogen and residual amounts of hydrocarbons in the gas are partial oxidized with oxygen in presence of a secondary reforming catalyst.

The primary and secondary steam reforming can in large scale methanol synthesis plants be replaced by autothermal reforming (ATR).

Recently, use of renewable energy in the methanol synthesis has become more available. As an example, combination of electrolysis of water operated on renewable energy, such as wind power and solar energy for the production of hydrogen by electrolysis of water and carbon dioxide from carbon capture or other carbon oxide source. The thus produced hydrogen and carbon dioxide are combined in stoichiometric ratios to form synthesis gas for methanol production.

The problem when using renewable energy in the methanol synthesis is that the supply of energy variates depending on the natural variations of for instance wind and sun. As a result, the flow of fresh methanol synthesis gas produced by means of renewable energy can variate substantially.

Because of relatively low single pass conversion rates of the methanol synthesis gas in the methanol reactor caused by equilibrium limitations, a large loop recycle stream of unconverted synthesis gas is required in the loop.

To substitute converted hydrogen and carbon oxides in the unconverted synthesis gas, a make-up gas of fresh synthesis gas must constantly be added into the loop recycle gas.

In case of large and frequent load variations because of variating flow of fresh synthesis gas to the synthesis loop, the mechanical stress caused by pressure variations introduced by the load variations will lead to unreferenced mechanical stress conditions that may cause mechanical failures of pressure bearing equipment. The temperature variations, however, will be limited.

Such operating conditions are especially relevant when the production is dependent on a variable flow of feedstock such as it is the case for green methanol production.

Traditionally a methanol loop does not feature a dedicated pressure control. In case of reduced feed flow to the loop, the loop pressure will drop. Thereby the conversion will reduce to a point eventually matching the make-up flow. In case of increasing feed flow, the pressure and the conversion will increase. Since the load of a traditional methanol plants tends to be stable over long periods, the absence of a pressure control does not normally represent a problem.

For a given reactor/loop configuration a possible way to control the loop pressure is variating the module M=(H2−CO2)/(CO+CO2) in the in the fresh synthesis gas, i.e. the make-up gas to decrease the reactivity of the gas. In some cases, it is also possible to vary the contents of inerts in the loop by reducing the purge flow, but this is seldom relevant for green methanol production where the make-up gas is very low in inerts. In practice, however, it is difficult to control loop pressure with this method.

We have found that the amount of feed gas to the methanol reactor can be controlled by the antisurge control of the recirculator (loop recycle compressor). The antisurge or kickback valve is typically a fast-reacting control element for protection against surge resulting in vibrations and thus damage of the compressor.

Pursuant to the above finding, preferred embodiments of present invention are the following:
1. Method for the control of pressure in a loop for the preparation of methanol comprising the steps of
(a) providing a fresh methanol synthesis gas;
(b) providing a loop recirculation gas;
(c) providing a loop recirculation compressor with an anti-surge valve and/or a compressor flow regulation valve;
(d) providing a methanol synthesis loop with one or more methanol reactors;
(e) recirculating the recirculation gas in the loop by means of the recirculation compressor;
(f) adding the fresh methanol synthesis gas into the loop recirculation gas;
(g) monitoring pressure in the methanol synthesis loop, wherein flow of the loop recirculation gas through the anti-surge valve and/or the recirculation compressor flow regulation valve is controlled to obtain a substantially constant pressure in the methanol synthesis loop.
2. The method of embodiment 1, wherein recycle compressor flow regulation valve is arranged in parallel with the antisurge valve.
3. The method of embodiment 2, wherein the flow through the antisurge valve or compressor flow regulation valve is directed to the cooling section of the methanol loop
4. The method of any one of embodiments 1 or 3, wherein flow of the fresh methanol synthesis gas is controlled by an antisurge valve of a compressor for the fresh synthesis gas.
5. The method of any one of embodiments 1 or 4, wherein flow of the fresh methanol synthesis gas is controlled by a pressure controller maintaining the suction pressure of a compressor for the fresh synthesis gas.
6. The method of any one of embodiments 1 to 5, comprising the further step of controlling temperature in a high-pressure loop separator arranged in the loop for the preparation of methanol.
7. The method of any one of embodiments 1 to 6, wherein hydrogen in the fresh methanol synthesis gas is provided by means of electrolysis of water.
8. The method of embodiment 7, wherein the electrolysis of water is performed in a solid oxide electrolysis cell.
9. The method of embodiments 1 to 6, wherein the fresh methanol synthesis gas is provided by co-electrolysis of water and carbon dioxide.
10. The method of any one of embodiments 1 to 9, wherein flow of the loop recirculation gas is additionally controlled by a loop pressure controller downstream or upstream the recirculation compressor.
11. The method of embodiment 10, wherein the loop pressure controller has load dependent tuning parameters.
12. The method of embodiment 10, wherein the loop pressure controller is configured in cascade with a flow control loop controlling the flow of unconverted synthesis gas recycled to the methanol reactor, the loop flow controller receives set point from the methanol loop pressure controller or from a calculation block which calculates the required recirculation flow based on fresh synthesis gas flow or preferably from a combination of the output of the mentioned loop pressure controller and mentioned calculation block.
13. The method of embodiment 10, wherein the loop flow controller receives a set point change from the power grid operator to make grid balancing service.
14. The method of embodiment 13, wherein the loop pressure controller operate the antisurge valve/compressor flow regulation valve and the loop flow controller in split range to obtain substantial constant loop pressure while ensuring sufficient flow to the recycle compressor.
15. The method of any one of embodiments 1 to 14, wherein the module of the fresh synthesis gas is controlled by a ratio controller of hydrogen and carbon oxides flow in the synthesis gas by controlling the carbon oxide flow rate relative to the hydrogen flow rate.
16. The method of embodiment 15, wherein the ratio controller of hydrogen to carbon oxides is compensated by a real-time analyzer.

For a green methanol plant with hydrogen produced by electrolysis from grid power the plant can supply critical grid balancing service without the need of large hydrogen buffer system in case the methanol production is able to be regulated as fast as required for grid balancing. This service needs to be available upon request from the grid in a matter of seconds when the frequency in the grid drops below a certain threshold value. The pressure in the methanol loop is maintained constant during grid balancing by a set point change of the loop pressure controller adjusting the methanol reactor flow when methanol plant is activated for grid balancing service and power input is reduced to new set point within seconds.

In case of slow load variation (days or weeks), the method according to the invention can be supplemented by control of the temperature in a high pressure methanol loop separator. Thereby reactivity of the loop recirculation loop gas flowing to the methanol reactor can be reduced when the concentration of methanol in the feed gas is increased. Higher temperature leads to less reactivity and higher loop pressure.

Thus, in a further step the temperature in a high pressure loop separator arranged in the loop for the preparation of methanol is controlled.

The loop separator separates liquid methanol product from the unconverted gas effluent from the synthesis converter at equilibrium between gas and liquid at the given pressure and temperature. At constant pressure and higher temperature gives higher content of product in unconverted gas to be recycled back to the methanol reactor. This will lower the potential conversion per pass since the synthesis reaction is limited by equilibrium resulting in reduced capacity of the synthesis loop at constant pressure.

One of the advantages of the invention is that energy for operating various equipment for the preparation of the synthesis gas can be renewable energy generated by windmills, solar cells, hydraulic energy or other renewables.

Preferably, the equipment comprises one or more electrolysis units, such as solid oxide electrolysis cells. The fast ramping up or down of feed rate to a green methanol plant, potentially results in several controllers becoming unstable because the PID controllers are typically tuned for conditions close to 100% capacity, and at lower production rates, say less than about 30%, a small disturbance in production causes a larger relative change in production.

Different controller tuning is required when operating at lower capacity. Therefore, to ensure that the control of the plant remains stable, these PID controllers need to have tuning parameters that vary with feed rate.

### DETAILED DESCRIPTION OF THE INVENTION

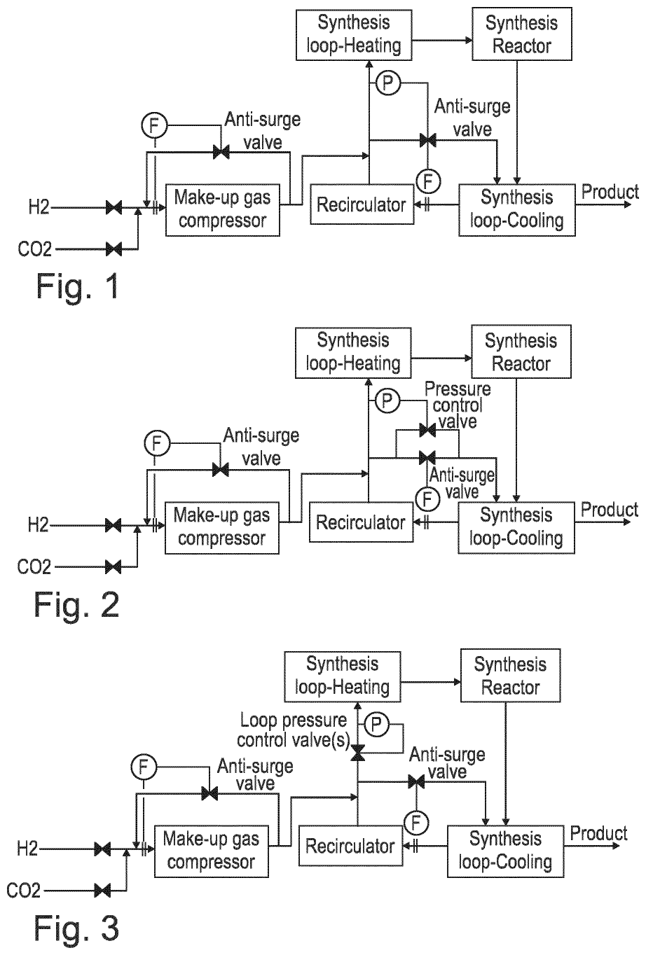

FIG. 1 shows a typical configuration of the make-up gas compressor, recirculator and synthesis loop.

If the antisurge valve is open, then less flow will pass on to the reactor. During start-up where the synthesis reactor is heated up by circulating gas in the loop then the antisurge will initially be fully open in order to protect the recirculator from surge and to reduce the flow rate to the reactor for easy control of the heating up phase.

The same valve (antisurge valve) is used simultaneously as compressor protection and flow control valve to the reactor. This is feasible as the two functions are never contradictory and in any case the machine protection will overrule all other set point to the valve. This concept is well proven for start-up of the synthesis.

Using renewable energy for production of synthesis gas will provide fluctuations throughout a day in feed gas flow rate resulting in many and possibly also abrupt synthesis pressure fluctuations. This can be smoothed out or even eliminated by the method according to the invention.

In normal operation, the recirculator antisurge valve can be used for control of the loop pressure. At full capacity the valve will remain closed and if less make up gas is available then the recirculation gas flow will be reduced correspondingly by controlled opening of the valve.

This will limit the conversion of synthesis gas in the loop to exactly the amount of make-up gas available resulting in keeping the same amount of gas in the loop and thus constant loop pressure.

There might be an understanding of the loop pressure is also controlled by the make-up compressor speed, but this is not the case as the make-up gas compressor will deliver the required pressure for a given conversion in the loop.

Since the method of the invention controls the conversion in the loop to maintain a constant loop pressure then the make-up gas compressor will follow the loop requirement. The only way the make-up gas compressor can do that and still be within its operating window (flow versus discharge pressure) is by opening its own antisurge valve(s) to compensate for the lower make-up gas flow available (see FIGS. 1 and 2).

There could be cases where it is not allowed to use the antisurge valve for loop pressure control valve. Then the alternative would be to install a control valve in parallel without jeopardizing the compressor surge protection as the antisurge valve opening is still governed by the compressor requirement measured as resulting flow from two control valves to the suction of the recirculator (see FIG. 2).

The flow through the compressor antisurge valve is normally returned to the suction side of the compressor through a dedicated antisurge cooler. Preferably this cooling can be done by the existing cooler in cooling section of the methanol loop having surplus capacity when less recycle flow is directed to the methanol reactor. At the same time a dedicated antisurge cooler can be avoided.

Since the conversion equilibrium temperature remains constant, a control which ensure the ratio between make-up gas and converter feed gas remains constant will nearly eliminate pressure and temperature fluctuations in the converter and loop.

Because the anti-surge valve has a security function, the flow from the compressor discharge side to suction side may additionally or completely be regulated by means of compressor flow regulation valve during feed gas flow variations.

The examples of FIGS. 1 and 2, will have a limitation on the turn down of the gas flow since the minimum flow to the methanol reactor will depend on the pressure drop ratio between the methanol reactor and the anti-surge valve.

FIG. 3 shows a configuration where the gas flow to the converter can be controlled down to a zero flow by means of a loop pressure controller and optionally a small bypass valve. When reducing or closing the loop pressure controller, the synthesis gas in the methanol reactor is retained in the reactor and maintains the reactor pressure. This will allow the loop pressure to be controlled down to very low load and still keep the loop pressure up and the converter in hot conditions. This is important in the case where suddenly the renewable energy and thus synthesis gas production comes back from low load to high load, then the conversion of synthesis gas into methanol can take place essentially instantaneous.

The example of FIG. 3 has a limitation on how fast the flow to the methanol reactor can be controlled.

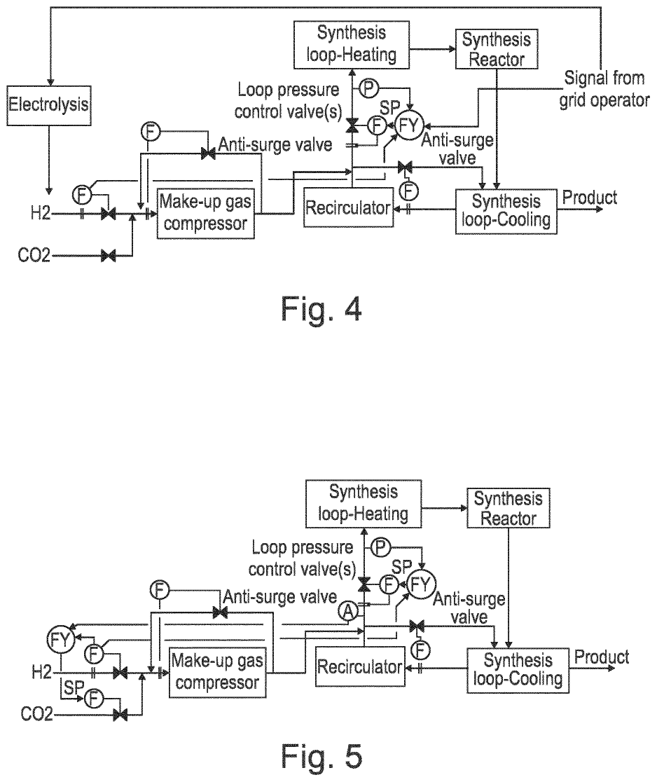

FIG. 4 shows a similar process layout as shown in FIG. 3, where the loop pressure controller receives a feed forward signal from the fluctuating make-up hydrogen flow to adjust the recirculation gas flow. Further the recirculation gas flow control can receive a signal from the power grid operatoradjusting the recirculation gas flow when methanol plant is activated for grid balancing service and power input is reduced to new set point within seconds.

The loop pressure controller can preferably control the recycle compressor antisurge valve/flow regulation valve and the loop flow regulation valve in split range and whereby dedicated flow regulation for the compressor can be avoided.

FIG. 5 shows a similar process layout as shown in FIG. 4, where one or more valves are foreseen to control recirculation gas flow, recirculator anti-surge flow, and make-up gas compressor anti-surge flow. The module of the make-up gas is controlled by a ratio controller of hydrogen and carbon dioxide flow in synthesis gas by controlling the carbon dioxide flow rate relative to the hydrogen flow rate. With many fluctuations perhaps daily in energy supply, and thus directly impacting the hydrogen flow rate and also the carbon dioxide flow rate, the measurement of hydrogen and carbon oxides flow might get a bit off set at each fluctuation. A small change in the make-up gas module will be amplified in the module of the loop recirculation gas and for this reason it is desirable to improve the module controller by having a near real-time analyzer on the make-up gas. Typically, a common gas chromatography analyzer is used for multiple sampling point leading to long tubing from each sampling point to the analyzer, which results in long cycle time for each analysis. Long cycle time of 10-20 min. is not suitable for adjustment of the module controller. A real-time analyzer can provide a cycle time of 10-20 sec. and the module controller can act in time before a wrong module gets amplified in the loop resulting in loss of capacity and/or pressure increase when high capacity is required.

In the figures,
P is pressure controller
F is flow controller
A is online/real time gas analyser (and controller) giving feedback signal to fresh make-up feed gas to adjust carbon oxides flow (if needed)
FY is a “calculation block” where modifications to a signal can be made, e.g. the FY at the H2 and CO2 feed gases receive a signal from the gas analyser and the hydrogen flow and calculates the required carbon oxides flow in order to get the desired H2/CO2 ratio in the loop. In the same way the calculation block in the loop receiving signal from the make-up gas, the pressure in the loop and the power grid and calculates the required circulation flow in order to have a stable pressure in the loop, when fresh make-up feed gas flow is either increased or decreased.

1. A method for controlling pressure in a loop for preparing methanol, the method comprising:
providing a fresh methanol synthesis gas;
providing a loop recirculation gas;
providing a loop recirculation compressor with an anti-surge valve and/or a compressor flow regulation valve;
providing a methanol synthesis loop with one or more methanol reactors;
recirculating the loop recirculation gas in the loop using the loop recirculation compressor;
adding the fresh methanol synthesis gas into the loop recirculation gas; and
monitoring pressure in the methanol synthesis loop,
wherein flow of the loop recirculation gas through the anti-surge valve and/or the compressor flow regulation valve is controlled to obtain a substantially constant pressure in the methanol synthesis loop.

2. The method of claim 1, further comprising providing a recycle compressor flow regulation valve in parallel with the anti-surge valve.

3. The method of claim 2, further comprising directing the flow of the loop recirculation gas through the anti-surge valve and/or the compressor flow regulation valve to a cooling section of the methanol synthesis loop.

4. The method of claim 1, further comprising controlling flow of the fresh methanol synthesis gas using an anti-surge valve of a compressor for the fresh methanol synthesis gas using an anti-surge valve of a compressor for the fresh methanol synthesis gas.

5. The method of claim 1, further comprising controlling flow of the fresh methanol synthesis gas using a pressure controller maintaining the suction pressure of a compressor for the fresh methanol synthesis gas.

6. The method of claim 1, further comprising controlling the temperature in a high-pressure loop separator arranged in the loop for preparing the methanol.

7. The method of claim 1, further comprising providing hydrogen in the fresh methanol synthesis gas by electrolysis of water.

8. The method of claim 7, wherein the electrolysis of water is performed in a solid oxide electrolysis cell.

9. The method of claim 1, wherein the fresh methanol synthesis gas is provided by co-electrolysis of water and carbon dioxide.

10. The method of claim 1, wherein the flow of the loop recirculation gas is additionally controlled using a loop pressure controller downstream or upstream of the loop recirculation compressor.

11. The method of claim 10, wherein the loop pressure controller has load dependent tuning parameters.

12. The method of claim 10, wherein:
the loop pressure controller is configured in cascade with a loop flow controller to control the flow of unconverted synthesis gas recycled to the one or more methanol reactors,
the loop flow controller receives a set point from:
the loop pressure controller or a calculation block configured to calculate a required recirculation flow based on fresh methanol synthesis gas flow, or
a combination of the outputs of the loop pressure controller and the calculation block.

13. The method of claim 12, wherein the loop flow controller receives a set point change from a power grid operator to balance grid service.

14. The method of claim 13, wherein the loop pressure controller is configured to control the anti-surge valve and/or the compressor flow regulation valve and the loop flow controller in split range to obtain substantially constant loop pressure while ensuring sufficient flow to a recycle compressor.

15. The method of claim 1, further comprising controlling flow of the fresh methanol synthesis gas into the loop recirculation gas using a ratio controller of hydrogen and carbon oxides flow in the fresh methanol synthesis gas by controlling a carbon oxide flow rate relative to a hydrogen flow rate.

16. The method of claim 15, wherein the ratio controller of hydrogen to carbon oxides is compensated by a real-time analyzer.
